(19) United States
(12) Patent Application Publication
(10) Pub. No.: US 2014/0195954 A1
(43) Pub. Date: Jul. 10, 2014

(54) Accessories as Workflow Priors in Medical Systems

(71) Applicant: Niraj K. Doshi, Knoxville, TN (US)

(72) Inventor: Niraj K. Doshi, Knoxville, TN (US)

(73) Assignee: SIEMENS MEDICAL SOLUTIONS USA, INC., Malvern, PA (US)

(21) Appl. No.: 14/147,770

(22) Filed: Jan. 6, 2014

Related U.S. Application Data
(60) Provisional application No. 61/750,415, filed on Jan. 9, 2013.

(51) Int. Cl. G06F 3/0484; G06F 3/0482; A61B 6/03; A61B 5/00; A61B 5/055

(52) U.S. Cl. CPC G06F 3/0484; A61B 5/0035; A61B 5/055; A61B 6/037; G06F 3/0482
USPC 715/771

(57) ABSTRACT

Workflow information is populated in a user interface of a medical system. One or more accessories are detected. The user interface is updated to assist the user based on the identity of the accessory. One or more lists may be created based on the identity, such as a list of examinations or protocols usable with the accessory. One or more fields for configuring the imaging system for scanning may be pre-filled based on the identity, such as using values used in a previous configuration for scanning with the accessory. The workflow through the user interface may be streamlined or configured to allow for more consistent, rapid, or easier input by the user based on the detection of the accessory.

### RELATED APPLICATIONS

The present patent document claims the benefit of the filing date under 35 U.S.C. §119(e) of Provisional U.S. Patent Application Ser. No. 61/750,415, filed Jan. 9, 2013, which is hereby incorporated by reference.

### BACKGROUND

The present embodiments relate to medical systems. In particular, accessories are used in medical systems.

Various imaging modalities utilize accessories that enhance the imaging. In ultrasound, optional accessories include transducers. In magnetic resonance imaging, the optional accessories include coils, such as local coils. In computed tomography (CT) or CT combined with positron emission tomography and CT(PET/CT) and single photon emission computed tomography (SPECT/CT) optional accessories include various injectors for performing contrast or other studies as well as CT simulation accessories such as a flat table top for radiation oncology simulation. Other accessories for any of various modes of medical imaging include heart monitors, breathing monitors, phantoms, and contrast agents.

The accessories are plugged into or wirelessly connect with the medical imaging system. These accessories may or may not be recognized by the medical imaging system. If the accessory is recognized, the accessory is generally used with no more than an acknowledgement of the accessory being displayed. In some cases, the accessories are just checked to verify that the accessory is plugged in correctly.

Once the accessory is connected or prior to connecting the accessory, the system operator generally selects the examination to be performed for a given patient. The system operator then fills out several fields in the user interface to prepare for performing the examination. This configuration process may occur several times repeatedly over the course of a day. Generally, the same study, using the same accessories, may be run several times, with the operator having to input the same parameters several times per day. This may introduce user input errors or minor changes in parameters, inadvertently. Also, there may be variations from user to user.

### BRIEF SUMMARY

By way of introduction, the preferred embodiments described below include methods, systems, and computer readable storage media for populating workflow information in a medical system. One or more accessories are detected. The user interface is updated to assist the user based on the identity of the accessory. One or more lists may be created based on the identity, such as a list of examinations or protocols usable with the accessory. One or more fields for configuring the imaging system for scanning may be pre-filled based on the identity, such as using values used in a previous configuration for scanning with the accessory. The workflow through the user interface may be streamlined or configured to allow for more consistent, rapid, or easier input by the user based on the detection of the accessory.

In a first aspect, a method is provided for populating workflow information in medical imaging. A connection of an accessory with a medical imaging system is detected. An identity of the accessory is identified in response to the detecting. The medical imaging system generates a user interface for configuring medical imaging by the medical imaging system. The user interface is configured for a protocol corresponding to the identity of the accessory.

In a second aspect, a system is provided for populating workflow information in medical imaging. A magnetic resonance imaging device includes an input, a display, and a processor. An accessory is connectable with the input of the medical scan device. The processor is configured to display a workflow with a protocol, fields of the protocol or the protocol and the fields of the protocol pre-filled based on detection of the accessory connected to the input.

In a third aspect, a non-transitory computer readable storage medium has stored therein data representing instructions executable by a programmed processor for populating workflow information in medical imaging. The storage medium includes instructions for recognizing an optional appliance to be used with a medical system, selecting a pre-determined workflow attributes based on the recognition of the optional appliance, and outputting the pre-determined workflow attributes.

The present invention is defined by the following claims, and nothing in this section should be taken as a limitation on those claims. Further aspects and advantages of the invention are discussed below in conjunction with the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a flow chart diagram of an example embodiment of a method for populating workflow information in a medical system;

FIGS. 2 and 3 are example user interfaces configured based on an identity of a connected accessory;

FIG. 4 is a block diagram of one embodiment of a system for populating workflow information in a medical system; and

FIG. 5 is a block diagram of one embodiment of a magnetic resonance system for populating workflow information in medical imaging based on detection of a type of coil.

### DETAILED DESCRIPTION OF THE DRAWINGS AND PRESENTLY PREFERRED EMBODIMENTS

Accessories are used to pre-determine or suggest attributes of the workflow for the medical system. Depending on the imaging or treatment modality, various accessories are used as part of the imaging study or therapy. The accessory is connected via a cable or wirelessly, such as an RFID or Bluetooth connection. In some cases, the accessories are recognized by the system, and checked if they are plugged in correctly. The accessory is identified from the connection. The identity or identities of the connected accessory or accessories suggests pre-determined examinations or studies. Additionally or alternatively, the knowledge of the accessory is used to fill in certain fields in the user interface. For example, based on historical data, workflow suggestions or examination parameters may be suggested or pre-filled for the operator to confirm.

Pre-filling fields and/or identifying relevant protocols may improve the efficiency and reduce the errors that may be caused by user input. Improving the workflow based on accessory identification may save time, reduce user input errors, and/or speed up the examination time.

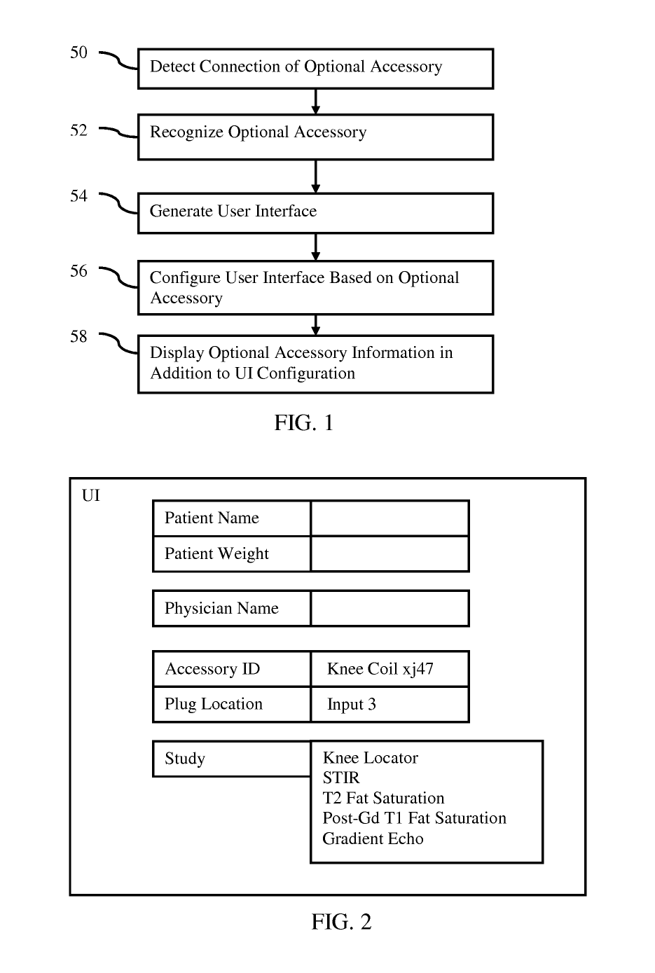

FIG. 1 shows one embodiment of a method for populating workflow information in a medical system. In general, detection of intent to use a particular accessory, such as by connection of the accessory, is used to assist the user input in the workflow. Protocols (e.g., studies or examinations) appropriate for the accessory may be presented to the user for selection. Fields associated with use of the accessory, such as values for scan parameters, may be pre-filled.

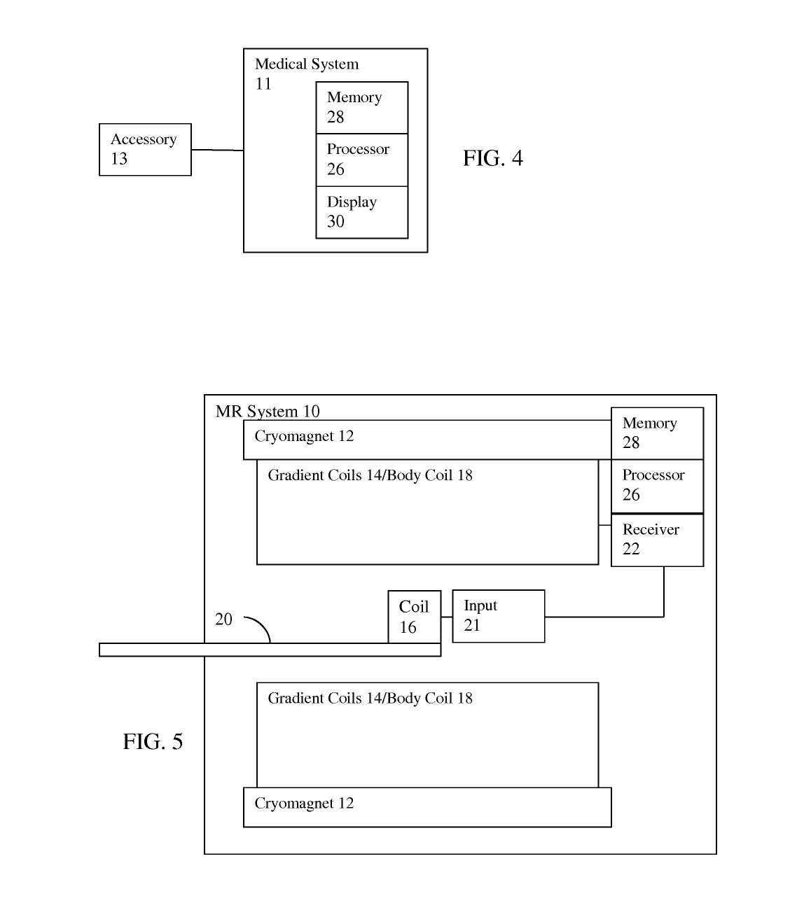

The method of FIG. 1 is performed by the medical system 11 of FIG. 4, the MR system 10 of FIG. 5, a different system, a processor, or a computer. For example, the medical system, an interface of the medical system, and/or a processor of the medical system detects connection, uses a memory or communications to recognize the accessory, generates the user interface, configures the user interface, and uses a display to display the user interface.

The medical system is a therapy system for treatment of a patient, an imaging system for scanning and visualizing the patient, or both. In one embodiment, the medical system is a combination of modalities, such as a positron emission and magnetic resonance imaging system.

The acts are performed in the order shown, but other orders may be provided. For example, the generating of the user interface in act 54 and the configuration of act 56 are performed simultaneously or as part of a general creation of the user interface for display. As another example, the display of act 58 is part of the generation of the user interface of act 54.

Additional, different, or fewer acts may be provided. For example, acts for operating the medical system based on the configuration from the user interface, acts associated with user interaction with the user interface, or both are provided.

In act 50, connection of an accessory is detected. The accessory is optional. The medical system may operate without the accessory, and/or the accessory is one option of various devices that may be used to for the medical system to operate. For example, an ultrasound system uses a transducer to operate. A transducer is needed to operation, but there are various options for type of transducer, so each transducer is optional. As another example, a magnetic resonance system may operate without any local coil. Each local coil is optional.

Any accessory may be provided. The accessory may be used to scan or acquire data for imaging. For example, the accessory is a local coil, such as a 4-channel flex coil (of any size), a special purpose coil (e.g., paddle coil), 6-channel body coil, 8-channel foot/ankle coil, 8-channel foot coil, 8-channnel head coil, 8-channel knee coil, 8-channel wrist coil, breast coil, head/neck coil, or any other local coils with any number of channels for any body part or parts. As another example, the accessory is an ultrasound transducer, such as a curved, linear, phased, 1.5D, multi-dimensional, or any other transducer with any number of elements, shape, and element spacing.

The accessory may be used to calibrate. The accessory is a phantom used for calibration of a computed tomography, positron emission tomography, single photon emission computed tomography, magnetic resonance or other imaging modality.

The accessory may be a transmitter for a therapeutic medical system. For example, the accessory is an acoustic transducer, electrode, aperture device, or probe used to apply energy to a patient.

The accessory may be a sensor ancillary to the scanning or therapy function of the medical system. For example, the accessory is an EKG system or electrode, a pulse-ox sensor, a breathing monitor, or other device for vital sign monitoring of the patient. These ancillary sensors may be used by the medical system to control scanning, imaging, and/or therapy.

The accessory connects with the medical system. The connection may be physical, such as by a cable and plug. The accessory is plugged into the medical system. For example, a local coil is plugged into a receptacle of a combination positron emission and magnetic resonance imaging system. Since the accessory is optional, the connection is releasable or not fixed.

Alternatively or additionally, the connection may be via communications. A wireless connection is provided. For example, RFID, Bluetooth, or Wi-Fi communications are established between the accessory and the medical system. The communications may be uni-directional or bi-directional. Using an exchange of information or mere receipt of information, a communications link is established between the accessory and the medical system. The communications link may be created in response to proximity (e.g., phantom within communications range), in response to user selection or activation of communications, automatically for any device within range, or using other processes.

The connection of the accessory to the medical system is detected. For plugs, electrical contact and/or communications over the link may be detected. For wireless communications, the creation of the link or the receipt of information may be detected. The accessory is detected either through initiation of physical connection or wireless connection. In other embodiments, the connection is detected by installation. For example, the accessory is a card or other component inserted into or electrically connected with the medical system. The installation is detected. The detection is of the accessory being connected, initial connection, or continuing to be connected.

The detection may occur without user input identifying the accessory. For example, the user plugs in the accessory, turns on the accessory within range, and/or activates communications with the accessory without otherwise indicating connection of the accessory. The medical system detects the accessory without the user inputting an identity of the accessory and/or without the user inputting that an accessory is being connected. In alternative embodiments, the user inputs to the medical system that an accessory is being connected, such as by selecting one of a list of available accessories.

Any number of accessories may be detected. For example, a single accessory is detected. As another example, two or more different types of accessories are detected. In yet another example, more than one of the same type of accessories is detected (e.g., two ultrasound transducers or two local coils).

In act 52, the accessory is recognized. When detected, the optional appliance to be used with the medical system is recognized. The detected connection indicates a possible intent to use the accessory with the medical system. Based on this intent, the identity of the accessory is determined.

The recognition is of an identity of the accessory. Any specificity of the identity may be provided. For example, a type of the accessory is identified. In the magnetic resonance example, that the local coil is a knee coil is identified. In the ultrasound transducer example, that the transducer is a linear array for abdominal scans is recognized. As another example, the identity is of the specific accessory. For example, a serial number or other coding distinguishing a given accessory for others even of the same type is determined. The serial number or other coding is the identity.

The recognition is performed by look-up. For example, a code or other communications is received by the medical system from the accessory. The medical system looks up the code in a table to determine the identity. The look-up table may be local and/or remote. For example, if a local table does not include the code, then the medical system may query a remote server for the identity based on the code. In other approaches, the accessory communicates the identity, such as providing the type information. Other techniques for recognition may be used, such as semi-automated approaches where the user selects or aids in recognizing the identity of the accessory.

In act 54, a user interface is generated. The medical system generates the user interface locally or remotely. Using software, hardware or both, the medical system causes display of graphics, images, or other screen outputs for user interaction. The user interface includes fields, check boxes, selectable buttons, selectable menus, tabs, or other software generated input devices for user interaction. Any navigation structure may be provided, such as including all information on one screen, including a hierarchal structure where one or more selections lead to other screens with appropriate or narrowed options. Tab structures may be used to configure different aspects or types of information with the user interface (e.g., tab for patient, tab for physician, and tab for scan).

The user interface is for configuring the medical system for operation. The configuration may be to scan a patient for imaging, to calibrate the medical system, and/or to apply therapeutic energy to the patient. The user interface is part of a workflow of the user for operating the medical system and/or for patient diagnosis or treatment. The medical system outputs, in the user interface, pre-determined workflow attributes, such as options relevant for the medical system and/or the patient. For example, options for scanning a patient are output as part of the user interface. The options are variables. In the magnetic resonance example, the options may include patient information (e.g., name and condition), physician information (e.g, name and contact information), accessory information, study information (e.g., type of scan or physicians orders), and characteristics of the scan (e.g., frequency, phase, or field of view). The attributes of the workflow include the optional protocols and/or values of parameters to be used in a given protocol.

In an alternative embodiment, the user interface is for a technician or other person performing quality control. For example, the user interface includes input options to be used for calibrating the medical system. Options related to the scan parameters, phantom, and technician are provided.

In act 56, the user interface is configured, at least in part, for workflow attributes corresponding to the identity of the accessory. Some aspect of the workflow represented in the user interface is varied or provided based on the identity. The protocol, such as selection of the protocol or values of protocol parameters, of the user interface includes information derived from the identity of the optional appliance. Pre-determined workflow attributes are provided in the user interface presented to the user based on the recognition of the accessory. The identity is used to initially set up the user interface or to later update the user interface. The configuration of the user interface and/or attributes of the user interface linked to the identity are stored in memory. The possible accessories are known, so the attributes of the workflow for a given recognized appliance may be looked-up as pre-determined values, lists, structures, or protocols. Alternatively, the prior determination is from a previous use of the same accessory or type of accessory.

The user interface is configured for a protocol. The protocol represents the work flow and/or a type of operation of the medical system. The study or examination to be performed and how (i.e., settings) to perform are attributes of the protocol.

Some aspect of the protocol is established based on the identity. For example, the navigation structure, the type of examination or study, the values used for scanning (e.g., values for scan parameters), or combinations thereof are selected by the medical system and incorporated into the user interface.

In one embodiment, the options available on the user interface are limited based on the identity. For example, a tab, hierarchal structure, or other aspect of navigation changes. The availability of an option may not be presented, such as not including tabs associated with breathing gating of scanning where an EKG monitor is recognized.

Other limiting of options may be provided. For example, specific fields for input by the user are not provided and other fields are provided. Fields that are not used with a given accessory are not provided, and fields that are used with that given accessory are provided in the user interface. For example, fields for configuring Doppler or color flow imaging are not provided for a B-mode scanning transducer.

The availability of selections may be conformed to the recognized accessory. A menu including different protocols, studies or examinations may be provided. For a medical imaging system, there may be tens or hundreds of different options for the types of scans to be performed. The identity of the accessory may be used to limit the available options. For example, a knee coil is identified as connected with the magnetic resonance imaging system. The list of options for the scanning protocol or study is limited to appropriate ones for the knee coil. FIG. 2 shows an example. The identity of the accessory and where the accessory is plugged in is included in the user interface. The study menu includes five optional studies that may be performed using the knee coil. Instead of being presented with options for brain, wrist, torso, or other studies, only studies associated with the detected knee coil are presented for selection by the user. The additional imaging protocols are not displayed.

In one embodiment, further limiting is provided. Rather than display options associated with the identified accessory, the previously selected option for that accessory is displayed. For example, if the knee coil were previously used for the STIR study, then only the STIR study is presented. The last used option, a list of X number of last used options, or any previously used options for that identity of the accessory are presented.

The user may be able to override the limitations. For example, one option may be an “other” so that the user can select a configuration not included in the limited options.

In another embodiment, values are included in one or more (e.g., at least two) fields based on the identity. As an alternative or in addition to limiting options for selection, the configuration of the user interface may provide suggested or possible values for one or more fields. The values for the fields are pre-filled. A single value is placed in each given field. Alternatively, two or more possible values are provided for user selection, such as in a drop down list, for one or more of the fields. The values are pre-determined in order to pre-fill the inputs.

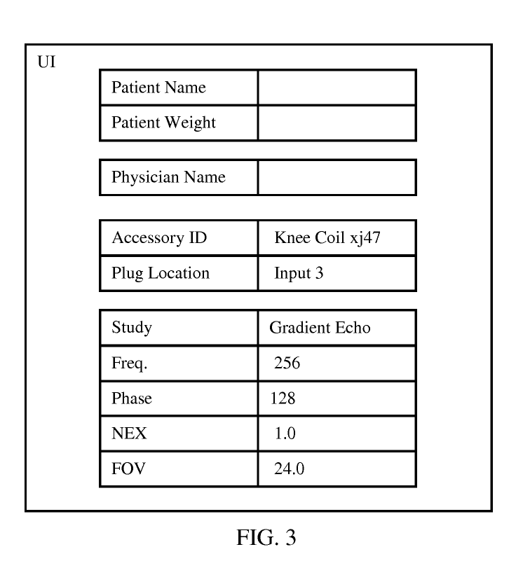

The values are based on the identity of the accessory. For example, a given accessory may only operate with a given value, such as being for a frequency or number of channels. This frequency or number of channels is provided as a pre-filled value in the user interface. As another example, previously used values for a given accessory or identity are pre-filled in the user interface. The settings most recently used or a default setting for the identity are output to the user as part of the user interface for confirmation, avoiding having to re-enter all of the values. The user merely changes any values as desired or leaves the pre-determined values. FIG. 3 shows an example. The study, frequency, phase, number of excitations (NEX), and field of view (FOV) values (e.g., 256, 128, 1.0, and 24.0 respectively) are output as pre-determined values in the user interface. The user may maintain the values or change the values.

The values used may be a function of other selections as well as the identity. For example, the user selects a study given a list, such as shown in FIG. 2. In response, the user interface is pre-filled with values for that type of study using the identified accessory (see FIG. 3). Alternatively, a single level is provided where any of the fields to be pre-filled are pre-filled without further user selection. For example, FIG. 3 represents an initial display of the user interface where the last imaging protocol (e.g., study) used and the corresponding values are pre-filled due to recognizing connection of the knee coil.

As shown in FIGS. 2 and 3, fields associated with the accessory itself are pre-filled. The identification and plug location are given as examples. The pre-filling and/or selection limitations are for other attributes of the workflow than the accessory itself. For example, the attributes include characteristics of the scan sequence or treatment sequence. The frequency, phase, NEX, and FOV are shown as scan characteristic examples, but any number of characteristics may be used. Some aspect controlling how the medical device uses the accessory is presented in the user interface based on the identity. Rather than just indicating the accessory, the scan values or type of scan are based on the identity.

Once the accessory is plugged in or connected, the medical system pre-selects frequently used or other pre-determined examination protocols or recalls the last exam protocol that was used in conjunction with that accessory (type of accessory or specific accessory). Certain fields in the GUI may be pre-filled based on the accessory used. This configuring of the user interface may minimize the time it takes for a particular patient to be registered on the system and the exam to be started.

In one embodiment, the user interface is for quality control. Values for calibration or other quality control protocols may be pre-filled. Available options may be limited based on the accessory. For example, a particular phantom or type of phantom is to be used for calibration. The phantom is queried or communicates with the medical system, providing recognition. The user interface is generated and configured with calibration options and/or pre-filled values based on the identity of the phantom.

Other accessory information may be presented to the user based on the identity in act 58. For example, an ownership of the accessory is presented. Passwords or other security information may be required to be input to allow use of the accessory. As another example, a half-life of a radioactive phantom or half-life related information may be presented. Since a phantom may have a half-life, this information may be used to order another phantom and/or to indicate to the user the viability of calibration using the current phantom. A day of creation or other measure of time remaining for reliable use of the phantom based on the identity of the specific phantom may be determined and displayed.

The user interface is displayed for interaction with the user. As the user interacts with the user interface as part of a workflow for diagnosis, imaging, or treatment of a patient, the user interface is updated. Each update may include all, some, or none of the list limitations and/or pre-fill values provided in response to recognition of the accessory.

In other embodiments, the user interface configuration varies based on the physician, technician, hospital, or medical entity. For example, an operator may have years of experience. More options associated with a given accessory may be presented to an experienced operator. Conversely, an inexperienced operator may be provided with a more limited option set or a single option. The most common option or options and associated pre-fill values are provided. As another example, different physicians may frequently order different examinations for a given identity. The options and/or pre-fill values may be different for different physicians even after being limited based on the recognized accessory.

FIG. 4 shows one embodiment of an accessory assisted system for populating workflow information in medical imaging. The system includes the medical system 11 and the accessory 13. Additional, different, or fewer components may be provided. For example, one or more additional accessories are provided. While the accessory 13 is shown with a cable connection, a wireless connection may be used in other embodiments. The same accessory 13 may be operable with any of various medical systems 11 or may be keyed to one particular medical system 11.

FIG. 5 shows an example of the system of FIG. 4, but where the medical system 11 is a magnetic resonance system 10 and the accessory 13 is a local coil 16.

The accessory 13 is necessary for operation of the medical system 11 or not necessary. For necessary, any one of a plurality of different accessories 13 may be used, so a given accessory 13 is optional. The accessory 13 is a sensor, transducer, coil, antenna, monitor, reader, or other device for measuring characteristics of the patient and/or applying energy to the patient. The accessory 13 may be a single device, may be an assembled collection of interacting devices, and/or may be a collection of separate devices.

The accessory 13 includes an antenna, RFID tag, RFID reader, circuit, wireless transmitter, wireless receiver, wireless transceiver, processor, dip switch, memory, switch network, or other arrangement for transmitting and/or receiving information. For example, a memory stores a code. Upon connection to the medical system 11, in response to a query from the medical system 11, or other trigger, the code is provided to the medical system. The medical system 11 may read the code or acquire the code from the accessory 13. The code identifies, such as by type and/or serial number, the type of accessory and/or the specific accessory.

The medical system 11 is of any modality. For example, the medical system is a magnetic resonance, an ultrasound, a computed tomography, x-ray, positron emission, single photon emission computed tomography, fluoroscopy, or other imaging system. As another example, the medical system 11 is a therapeutic system, such as an ultrasound, x-ray, radiation, particle, or other therapy system.

The medical system 11 includes a processor 26, a memory 28, and a display 30. Additional, different or fewer components may be provided. For example, a network interface is provided. As another example, a user input (e.g., keyboard, mouse, trackball, soft buttons, hard buttons, and/or touch pad or screen) is provided. In general, the memory 28 stores previously used, programmed, or other pre-determined user interface attributes associated with different accessories 13. The display 30 displays the user interface to the user. The processor 26 controls configuration of the user interface, recognition of the accessory 13 and associating the accessory 13 with user interface attributes.

The display 30 is a monitor, CRT, LCD, plasma, projection, touch screen, printer, or other display for outputting a user interface. The display 30 is fed images from a buffer. The images include user workflow information, such as the fields and/or input options shown in FIGS. 2 and 3.

Referring the example of FIG. 5, the system 10 includes a cryomagnet 12, gradient coils 14, whole body coil 18, local coil 16, patient bed 20, MR receiver 22, processor 26, and memory 28. Additional, different, or fewer components may be provided. For example, another local coil or surface coil is provided. Other optional appliances may be connected or provided for connection, such as breathing or cardiac monitors.

The local coil 16 is an accessory 13. The local coil 16 includes a cable and plug for connecting with a receptacle of the MR system 10, such as a receptacle on an input 21. The input 21 is an interface for connecting with one or more accessories. The input 21 may mate with the accessory, such as having a receptacle or plug matching the plug or receptacle of the local coil 16. The input 21 is located on the patient bed 20, but may be in other locations. In other embodiments, the input 21 is a wireless card or interface. The local coil 16 is connectable with the MR system 10, such as using the plug or wireless linking. To arrange for operation of the MR system 10, the local coil 16 is connected. In preparation for operation, the connection may be initiated. This occurs automatically based on proximity or signal reception, or based on powering on. In other embodiments, the connection is initiated in response to user activation, such as plugging in the accessory.

Other parts of the MR system are provided within a same housing, within a same room (e.g., within the radio frequency cabin), within a same facility, or connected remotely. The other parts of the MR portion may include cooling systems, pulse generation systems, image processing systems, and user interface systems. Any now known or later developed MR imaging system may be used with the modifications discussed herein. The location of the different components of the MR system is within or outside the radio frequency (RF) cabin, such as the image processing, tomography, power generation, and user interface components being outside the RF cabin. Power cables, cooling lines, and communication cables connect the pulse generation, magnet control, and detection systems within the RF cabin with the components outside the RF cabin through a filter plate.

The processor 26 and memory 28 are part of a medical imaging system, such as the MR system 10. In one embodiment, the processor 26 and memory 28 are part of the MR receiver 22. Alternatively, the processor 26 and memory 28 are part of an archival and/or image processing system, such as associated with a medical records database workstation or server. In other embodiments, the processor 26 and memory 28 are a personal computer, such as desktop or laptop, a workstation, a server, a network, or combinations thereof. The processor 26 and memory 28 may be provided without other components for implementing the method.

The magnetic resonance scanner includes the cryomagnet 12, gradient coils 14, body coil 18, and any local coils 16. The cryomagnet 12, gradient coils 14, and body coil 18 are in the RF cabin, such as a room isolated by a Faraday cage. A tubular or laterally open examination subject bore encloses a field of view. A more open arrangement may be provided. The patient bed 20 (e.g., a patient gurney or table) supports an examination subject such as, for example, a patient with a local coil arrangement, including the coil 16. The patient bed 20 may be moved into the examination subject bore in order to generate images of the patient. Any local coils 16 are placed on, under, against, or in the patient. The local coil 16 may be attached to the bed 20. Received signals may be transmitted by the local coil arrangement to the MR receiver 22 via, for example, coaxial cable or radio link (e.g., via antennas) for localization.

In order to examine the patient, different magnetic fields are temporally and spatially coordinated with one another for application to the patient. The cryomagnet 12 generates a strong static main magnetic field B₀ in the range of, for example, 0.2 Tesla to 3 Tesla or more. The main magnetic field B₀ is approximately homogeneous in the field of view.

The gradient coils 14 radiate magnetic gradient fields in the course of a measurement in order to produce selective layer excitation and for spatial encoding of the measurement signal. The gradient coils 14 are controlled by a gradient coil control unit that, like the pulse generation unit, is connected to the pulse sequence control unit.

The nuclear spins of atomic nuclei of the patient are excited via magnetic radio-frequency excitation pulses that are transmitted via a radio-frequency antenna, shown in FIG. 5 in simplified form as a whole body coil 18, and/or possibly a local coil arrangement (e.g., local coil 16). In the described embodiments, the method is applied to a water proton signal. The technique may also be applied to other body compartments like blood, fat and also to other MR-visible nuclei like 31P, 19F and 23Na. The body coil 18 is a single-part or includes multiple coils. Radio-frequency excitation pulses are generated, for example, by a pulse generation unit controlled by a pulse sequence control unit. After being amplified using a radio-frequency amplifier, the radio-frequency excitation pulses are routed to the body coil 18 and/or local coils 16. The signals are at a given frequency band. For example, the MR frequency for a 3 Tesla system is about 125 MHz +/−500 KHz. Different center frequencies and/or bandwidths may be used.

Any pulse sequence may be used for scanning. The scanning occurs before adding contrast agent for a baseline measure and after or during adding of contrast agent. The scanning may be used just with contrast agent or only without contrast agent.

The signals emitted by the excited nuclear spins are received by the local coils 16 and/or the body coil 18. In the course of an MR measurement, the excited nuclei induce a voltage in the local coils 16. In some MR tomography procedures, images having a high signal-to-noise ratio (SNR) may be recorded using local coil arrangements (e.g., loops, local coils 16). The local coil arrangements (e.g., antenna systems) are disposed in the immediate vicinity of the examination subject on (anterior) or under (posterior) or in the patient. The received signals are amplified by associated radio-frequency preamplifiers and transmitted in analog or digitized form.

The MR receiver 22 connects with the coil or coils 16. The connection is wired (e.g., coaxial cable) or wireless. The connection is for data from the coils 16 to be transmitted to and received by the MR receiver 22. The data is K-space data. In response to an MR pulse, the coils 16 receive signals as the K-space data and transmit the data to the MR receiver 22. Any pulse sequence may be used. Any spatial resolution may be provided.

The signals are processed further and digitized by the MR receiver 22. The recorded measured data is stored in digitized form as complex numeric values in a k-space matrix. An associated MR image of the examination subject may be reconstructed using a one or multidimensional Fourier transform from the k-space matrix populated with values. Each MR image is a frame of complex or phase sensitive values.

The MR receiver 22 includes the processor 26 or another processor (e.g., digital signal processor, field programmable gate array, or application specific circuit for applying an inverse Fourier transform) for reconstructing the K-space data. The MR receiver 22 is configured by hardware or software to calculate data in the spatial domain from the K-space data. The processor 26 applies an inverse Fast Fourier transform to calculate the power spectrum of the projection data. The power spectrum provides intensity as a function of frequency. The frequency corresponds to space or distance. The MR data as acquired is a function of frequency and after applying inverse FT becomes a function of space. Any transform for reconstructing spatial data from the K-space data may be used.

The MR system 10 and MR scanner are configured by hardware and/or software to acquire frames of data. Scans are performed to acquire frames of data representing the tissue response. The scans are defined various variables for which different settings are available.

The processor 26 is a general processor, central processing unit, control processor, graphics processor, digital signal processor, three-dimensional rendering processor, image processor, application specific integrated circuit, field programmable gate array, digital circuit, analog circuit, combinations thereof, or other now known or later developed device for populating a user interface of a workflow. The processor 26 is a single device or multiple devices operating in serial, parallel, or separately. The processor 26 may be a main processor of a computer, such as a laptop or desktop computer, or may be a processor for handling some tasks in a larger system, such as being part of the MR receiver 22 or MR imaging system 10. The processor 26 is configured by instructions, design, hardware, and/or software to perform the acts discussed herein.

The processor 26 is configured to display user interface as part of a workflow. The workflow includes one or more protocols, fields of the protocol, or both. The protocols are displayed as a single selected protocol or as multiple optional protocols. The fields are ones appropriate for the patient, technician, physician, and MR system 10. The workflow is represented, at least in part, by a menu, screen, or inputs for configuring the MR system 10 to scan the patient.

The processor 26, in response to signals from the input 21, detects the local coil 16. The type or specific local coil 16 is determined. For example, the processor 26 queries the local coil 16 or receives from the local coil 16 an identifier. The identifier is used to look up in the memory 28 further identification, such as a type. Alternatively or additionally, the identifier is used to look up user interface attributes associated with the identifier. Based on the identity, the processor 26 configures the user interface. The user interface is pre-filled. The workflow may be displayed with a protocol or list of protocols specific to the connected local coil 16. The processor 26 selects protocols that may be used with the local coil 16 and does not select protocols that cannot be used with the local coil 16. The selected protocols are displayed, such as in a drop down menu or list.

The processor 26 alternatively or additionally pre-fills one or more fields in the user interface for the workflow. The fields may be for any purpose, such as fields identifying the local coil 16 and characteristics of the local coil (e.g., number of channels, type, and connected input 21). The fields may be for use of the local coil 16 in scanning, such as frequency and phase. One, multiple, or all characteristics of the scan sequence for operation of the MR system 10 with the local coil 16 are pre-filled by the processor 26 based on the detection of the local coil 16. Any now known or later developed variables for defining the scan may be used. The navigation structure of the user interface may be varied depending on the detected local coil 16.

The processor 26 is configured to perform the method of FIG. 1 or another method. The processor 26 performs the acts, controls other components (e.g., MR system 10) to perform the acts, and/or acquires data to perform the acts of FIG. 1. For example, the processor 26 controls the input 21 to acquire identity information, such as a code from the local coil 16. The processor 26 looks up any further identity information from the memory 28 and configures the user interface according to a pre-determined format, list, and/or values for settings for the connected local coil 16. The processor 26 causes the display 30 to output the user interface.

The memory 28 is a graphics processing memory, video random access memory, random access memory, system memory, cache memory, hard drive, optical media, magnetic media, flash drive, buffer, database, combinations thereof, or other now known or later developed memory device for storing data or video information. The memory 28 is part of an imaging system, part of a computer associated with the processor 26, part of a database, part of another system, a picture archival memory, or a standalone device.

The memory 28 stores previously used configurations of workflow by accessory or type of accessory. Alternatively or additionally, depending on user setup, the memory 28 stores common, pre-determined, expert arranged, or other user interface attributes appropriate for each of a plurality of accessories. A set of user interface attributes (e.g., navigation structure, protocol or lists of protocols, and/or values for fields configuring the scanning) is stored with reference to a corresponding accessory or combination of accessories. A different set is provided for each accessory or combination of accessories. Alternatively, the attributes are coded such that attributes associated with different accessories that may be connected at a same time may be combined in one user interface. Any conflicts may be resolved using a priority ranking or by including options in a list.

The memory 28 or other memory is alternatively or additionally a non-transitory computer readable storage medium storing data representing instructions executable by the programmed processor 26 for populating workflow information in medical imaging. The instructions for implementing the processes, methods and/or techniques discussed herein are provided on non-transitory computer-readable storage media or memories, such as a cache, buffer, RAM, removable media, hard drive or other computer readable storage media. Non-transitory computer readable storage media include various types of volatile and nonvolatile storage media. The functions, acts or tasks illustrated in the figures or described herein are executed in response to one or more sets of instructions stored in or on computer readable storage media. The functions, acts or tasks are independent of the particular type of instructions set, storage media, processor or processing strategy and may be performed by software, hardware, integrated circuits, firmware, micro code and the like, operating alone, or in combination. Likewise, processing strategies may include multiprocessing, multitasking, parallel processing, and the like.

In one embodiment, the instructions are stored on a removable media device for reading by local or remote systems. In other embodiments, the instructions are stored in a remote location for transfer through a computer network or over telephone lines. In yet other embodiments, the instructions are stored within a given computer, CPU, GPU, or system.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I (we) claim:

1. A method for populating workflow information in medical imaging, the method comprising:
detecting connection of an accessory with a medical imaging system;
identifying an identity of the accessory in response to the detecting;
generating, by the medical imaging system, a user interface for configuring medical imaging by the medical imaging system; and
configuring the user interface for a protocol corresponding to the identity of the accessory.

2. The method of claim 1 wherein detecting comprises establishing a communications link between the accessory and the medical imaging system.

3. The method of claim 1 wherein detecting comprises detecting the accessory being plugged into the medical imaging system.

4. The method of claim 1 wherein detecting comprises detecting the accessory without user input identifying the identity of the accessory.

5. The method of claim 1 wherein identifying comprises identifying a type of the accessory as the identity.

6. The method of claim 1 wherein identifying comprises identifying a serial number of the accessory as the identity.

7. The method of claim 1 wherein generating the user interface comprises displaying options for scanning a patient, and wherein configuring comprises limiting the options based on the identity of the accessory.

8. The method of claim 7 wherein displaying the options comprises displaying a list of two or more imaging protocols, and wherein limiting the options comprises not displaying additional imaging protocols.

9. The method of claim 1 wherein generating the user interface comprises displaying fields for user input in a workflow for the medical imaging, and wherein configuring comprises including values in at least two of the fields, the values being a function of the identity of the accessory.

10. The method of claim 9 wherein including the values comprises pre-filling the fields for a scan sequence, the values comprising characteristics of the scan sequence.

11. The method of claim 1 wherein the accessory comprises a phantom, wherein generating the user interface comprises generating a quality control user interface for the medical imaging, and wherein configuring comprises pre-filling values of the quality control user interface based on the identity of the phantom.

12. The method of claim 1 further comprising:
displaying half-life, ownership, or half-life and ownership information for the accessory based on the identity.

13. The method of claim 1 wherein detecting comprises detecting a coil of a magnetic resonance portion of a combination magnetic resonance and positron emission imaging system, and wherein configuring comprises displaying a menu including the protocol, the menu being based on the identity of the coil and the protocol including both magnetic resonance and positron emission scan configurations.

14. The method of claim 1 wherein configuring comprises displaying the protocol as a last used protocol for the accessory with pre-filled fields from the last used protocol.

15. A system for populating workflow information in medical imaging, the system comprising:
a magnetic resonance imaging device comprising:
an input;
a display; and
a processor; and
an accessory connectable with the input of the medical resonance imaging device;
wherein the processor is configured to display a workflow with a protocol, fields of the protocol or the protocol and the fields of the protocol pre-filled based on detection of the accessory connected to the input.

16. The system of claim 15 wherein the accessory comprises a coil with a plug matable with the input, and wherein the processor is configured to display the workflow as the protocol specific to a type of the coil.

17. The system of claim 15 wherein the accessory comprises a coil with a plug matable with the input, and wherein the processor is configured to display the workflow with a list of protocols including the protocol, the list being selected by the processor based on an identity of the coil.

18. The system of claim 15 wherein the accessory comprises a coil, and wherein the processor is configured to pre-fill the fields with characteristics of a scan sequence for operation of the magnetic resonance imaging device using the coil.

19. In a non-transitory computer readable storage medium having stored therein data representing instructions executable by a programmed processor for populating workflow information in a medical system, the storage medium comprising instructions for:
recognizing an optional appliance to be used with the medical system;
selecting pre-determined workflow attributes based on the recognition of the optional appliance; and
outputting the pre-determined workflow attributes.

20. The non-transitory computer readable storage medium of claim 19 wherein selecting the pre-determined workflow attributes comprises selecting a study, selecting values for scan parameters, or selecting both the study and the values for the scan parameters.
